(19) United States
(12) Patent Application Publication
(10) Pub. No.: US 2014/0004530 A1
(43) Pub. Date: Jan. 2, 2014

(54) METHOD FOR THE EVALUATION OF COMPOUND-TARGET INTERACTIONS ACROSS SPECIES

(71) Applicants: Marcus Bantscheff, Dielheim (DE); Mikhail Savitski, Heidelberg (DE)

(72) Inventors: Marcus Bantscheff, Dielheim (DE); Mikhail Savitski, Heidelberg (DE)

(73) Assignee: CELLZOME GMBH, Heidelberg (DE)

(21) Appl. No.: 13/993,221

(22) PCT Filed: Dec. 22, 2011
(86) PCT No.: PCT/EP11/06503

§ 371 Date: Aug. 30, 2013

Related U.S. Application Data
(60) Provisional application No. 61/498,744, filed on Jun. 20, 2011.

(30) Foreign Application Priority Data
Dec. 22, 2010 (EP) 10015931.8

(51) Int. Cl. G01N 33/68

(52) U.S. Cl. CPC G01N 33/6848
USPC 435/7.4; 436/501; 435/7.1

(57) ABSTRACT

The present invention relates to a method for the determination of the species profile of a protein-interacting compound, comprising the steps of a) providing a protein preparation derived from one species (species-1) containing a species-1-variant of said protein, b) providing a protein preparation derived from another species (species-2) containing a species-2-variant of said protein, c) mixing said preparations, d) contacting said mixed protein preparations with a ligand of said protein and with a given protein-interacting compound under conditions allowing the formation of a complex between said ligand and said protein, and e) detecting the complexes formed in step d) with the help of mass spectrometry.

The goal of drug discovery is to develop safe and effective medicines. In order to achieve this goal, translational preclinical animal models are important to predict the therapeutic potential of newly developed drugs in humans. Typically, the efficacy and safety of new drug candidates is evaluated in several animal species. For the interpretation of these studies it is important to know whether the drug interacts and modulates the intended target (e.g. a receptor protein or enzyme) in the same way in the animal model as in humans. For example, there can be significant species differences in the amino acid sequence of the drug target and consequently in the affinity for the drug leading to different pharmacological effects.

For several G-protein coupled receptors (GPCRs) significant species differences are known and have complicated drug discovery efforts. Various species orthologs of the histamine H₄ receptor (H₄R) were cloned based on their sequence homology to the human H₄R, including mouse, rat, guinea pig, monkey (Macaca fascicularis) and dog. These H₄R orthologs display significant differences in the affinity for the endogenous agonist histamine and synthetic ligands. One approach to investigate these differences is to express the H₄Rs of different species in the HEK293T cell line by transient transfection of expression vectors and to measure the interaction of the expressed H₄Rs with ligands. The results show species differences up to 10-fold in the binding to histamine. It should be noted that differences in the expression levels and method of expression may lead to variability in the measured pharmacological values (Lim et al., 2010. Mol. Pharmacol. 77(5):734-743).

For other drug target classes, for example kinases, interspecies sequence differences are typically more subtle than for GPCRs such as H₄Rs. Consequently, small differences in the affinity for inhibitors are more difficult to assess, especially when recombinant enzymes or active fragments thereof are used in conventional enzyme assays.

Chemical proteomic methods using endogenously expressed enzymes have been used to study the interaction of inhibitors with their protein targets in different species (Bantscheff et al., 2007, Nat Biotechnol. 25(9): 1035-1044; WO 2006/134056 A1; WO 2009/098021 A1). In this approach, cell lysates generated from cell lines or tissues of different species are used as biological material and the interaction of small molecule compounds with their protein targets is analysed by mass spectrometry or immunodetection methods.

One prerequisite for the detection of small interspecies differences of compound-target interactions, and thereby determining the species profile of a given compound of interest, is to minimize experimental variability such as, for example inter-assay variability. For example, small interspecies differences of drug-target interactions may have significant pharmacological effects and are important to consider when interpreting and extrapolating the results of animal studies. Another aspect is the use of unmodified endogenously expressed proteins because the use of recombinant proteins may cause additional experimental differences.

The profiling of compound-target interactions including the analysis of their binding affinities to particular targets have been described in the art (see, e.g., Bantscheff et al., 2007, Nature Biotechnology, Vol. 25(9), pp. 1035-1044; and Bantscheff et al., 2007, Ernst Schering Foundation Symposium Proceedings, Vol. 3, pp. 1-28). However, in these studies separate experiments were performed to characterize the compound-target interactions in different species and the results of these separate experiments were compared with the disadvantage of inter-assay variability.

In view of the above, there is a need for improved methods for the determination of interspecies differences of compound-target interactions which are generally understood to define the species profile of a particular compound of interest.

In a first aspect, the present invention provides a method for the determination of the species profile of a protein-interacting compound, comprising the steps of
a) providing a protein preparation derived from one species (species-1) containing a species-1-variant of said protein,
b) providing a protein preparation derived from another species (species-2) containing a species-2-variant of said protein,
c) mixing said protein preparations,
d) contacting said mixed protein preparations with a ligand of said protein and with a given protein-interacting compound under conditions allowing the formation of a complex between said ligand and said protein, and
e) detecting the complexes formed in step d) with the help of mass spectrometry.

In the context of the present invention, it has been found that the mixing of protein preparations like cell lysates derived from different species and subsequent deconvolution of orthologous proteins based on species-specific peptides enables the reliable detection of interspecies differences in the affinity of a given compound for a protein target of interest. The orthologs are identified by mass spectrometry using a targeted data acquisition approach based on quantifying species-specific peptides.

According to the invention, orthologs and paralogs are two types of homologous sequences. Orthology describes genes (and encoded proteins) in different species that derive from a common ancestor. Orthologous genes may or may not have the same function. Paralogy describes homologous genes (and encoded proteins) within a single species that diverged by gene duplication (Koonin, 2005. Annu. Rev. Genet. 39:309-338).

In the context of the present invention the term “species profile” means a selectivity profile of a protein-interacting compound in at least two different species. The term “species profile” includes any information which characterizes the compound-protein interaction such as affinity (for example IC₅₀ and K_{D} values) and ratios thereof which describe selectivity in the different species. In the context of the present invention, the species profile preferably refers to interspecies differences including, for example, the identification and quantification of species-specific peptides derived from orthologous proteins.

According to the invention, first a protein preparation derived from one species (species-1) containing a species-1-variant of a given protein and a protein preparation derived from another species (species-2) containing a species-2-variant of said protein are provided.

Preferably, said protein is an enzyme.

According to the invention, the given enzyme may be any enzyme including, but not limited to, kinases, histone acetyltransferases, histone deacetylases, histone methylases and histone demethylases.

The methods of the present invention can be performed with any protein preparation as a starting material, as long as the protein is solubilized in the preparation. Examples include a liquid mixture of several proteins, a cell lysate, a partial cell lysate which contains not all proteins present in the original cell or a combination of several cell lysates, in particular in cases where not every target protein of interest is present in every cell lysate. The term “protein preparation” also includes dissolved purified protein.

The presence of the protein in a protein preparation of interest can be detected on Western blots probed with antibodies that are specifically directed against said protein.

Cell lysates or partial cell lysates can be obtained by isolating cell organelles (e.g. nucleus, mitochondria, ribosomes, golgi etc.) first and then preparing protein preparations derived from these organelles. Methods for the isolation of cell organelles are known in the art (Chapter 4.2 Purification of Organelles from Mammalian Cells in “Current Protocols in Protein Science”, Editors: John E. Coligan, Ben M. Dunn, Hidde L. Ploegh, David W. Speicher, Paul T. Wingfield; Wiley, ISBN: 0-471-14098-8).

In addition, protein preparations can be prepared by fractionation of cell extracts thereby enriching specific types of proteins such as cytoplasmic or membrane proteins (Chapter 4.3 Subcellular Fractionation of Tissue Culture Cells in “Current Protocols in Protein Science”, Editors: John E. Coligan, Ben M. Dunn, Hidde L. Ploegh, David W. Speicher, Paul T. Wingfield; Wiley, ISBN: 0-471-14098-8).

Furthermore protein preparations from body fluids can be used (e.g. blood, cerebrospinal fluid, peritoneal fluid and urine).

For example, whole embryo lysates derived from defined development stages or adult stages of model organisms such as C. elegans can be used. In addition, whole organs such as heart dissected from mice can be the source of protein preparations. These organs can also be perfused in vitro in order to obtain a protein preparation.

In a preferred embodiment of the methods of the invention, the provision of a protein preparation includes the steps of harvesting at least one cell containing the protein, preferably the enzyme, and lysing the cell.

Suitable cells for this purpose are, e.g., those cells or tissues in which the enzyme of interest is expressed. For example, members of the PI3K kinase family are expressed in most cells and tissues. PI3K gamma (PI3Kγ; PIK3CG) is preferentially expressed in cells of the hematopoietic system (e.g. granulocytes, macrophages, mast cells and platelets) but also in cardiomyocytes, vascular smooth muscle and vascular epithelium cells. PI3K delta (PI3Kδ; PIK3CD) is ubiquitously expressed with pronounced expression in lymphocytes, granulocytes and mast cells.

Therefore, in a preferred embodiment, cells isolated from peripheral blood represent a suitable biological material. Procedures for the preparation and culture of human lymphocytes and lymphocyte subpopulations obtained from peripheral blood (PBLs) are widely known (W. E Biddison, Chapter 2.2 “Preparation and culture of human lymphocytes” in Current Protocols in Cell Biology, 1998, John Wiley & Sons, Inc.). For example, density gradient centrifugation is a method for the separation of lymphocytes from other blood cell populations (e.g. erythrocytes and granulocytes). Human lymphocyte subpopulations can be isolated via their specific cell surface receptors which can be recognized by monoclonal antibodies. The physical separation method involves coupling of these antibody reagents to magnetic beads which allow the enrichment of cells that are bound by these antibodies (positive selection). The isolated lymphocyte cells can be further cultured and stimulated by adding antibodies directed against the T-cell receptor or co-receptors such as CD-3 to initiate T-cell receptor signaling and subsequently phosphorylation of downstream kinases such as members of the PI3K family (Houtman et al., 2005, The Journal of Immunology 175(4), 2449-2458).

As an alternative to primary human cells cultured cell lines (e.g. MOLT-4 cells or rat basophilic leukemia (RBL-2H3) cells) can be used. RBL-2H3 cells can be stimulated by cross-linking the high-affinity receptor for IgE (FcepsilonRI) by multivalent antigens to induce activation of PI3K (Kato et al., 2006, J. Immunol. 177(1): 147-154).

In a preferred embodiment, the cell is part of a cell culture system and methods for the harvest of a cell out of a cell culture system are known in the art (literature supra).

The choice of the cell will mainly depend on the expression of the given protein, preferably the given enzyme, since it has to be ensured that the protein is principally present in the cell of choice. In order to determine whether a given cell is a suitable starting system for the methods of the invention, methods like Westernblot, PCR-based nucleic acids detection methods, Northernblots and DNA-microarray methods (“DNA chips”) might be suitable in order to determine whether a given protein of interest is present in the cell.

The choice of the cell may also be influenced by the purpose of the study. If the in vivo efficacy for a given drug needs to be analyzed, then cells or tissues may be selected in which the desired therapeutic effect occurs (e.g. granulocytes or mast cells). By contrast, for the elucidation of protein targets mediating unwanted side effects the cell or tissue may be analysed in which the side effect is observed (e.g. cardiomycytes, vascular smooth muscle or epithelium cells).

Furthermore, it is envisaged within the present invention that the cell containing the given protein, preferably the given enzyme, may be obtained from an organism, e.g. by biopsy. Corresponding methods are known in the art. For example, a biopsy is a diagnostic procedure used to obtain a small amount of tissue, which can then be examined miscroscopically or with biochemical methods. Biopsies are important to diagnose, classify and stage a disease, but also to evaluate and monitor drug treatment.

It is encompassed within the present invention that by the harvest of the at least one cell, the lysis is performed simultaneously. However, it is equally preferred that the cell is first harvested and then separately lysed.

Methods for the lysis of cells are known in the art (Karwa and Mitra: Sample preparation for the extraction, isolation, and purification of Nuclei Acids; chapter 8 in “Sample Preparation Techniques in Analytical Chemistry”, Wiley 2003, Editor: Somenath Mitra, print ISBN: 0471328456; online ISBN: 0471457817). Lysis of different cell types and tissues can be achieved by homogenizers (e.g. Potter-homogenizer), ultrasonic disintegrators, enzymatic lysis, detergents (e.g. NP-40, Triton® X-100, CHAPS, SDS), osmotic shock, repeated freezing and thawing, or a combination of these methods.

According to the methods of the invention, the protein preparation containing the protein is contacted with a ligand for the protein immobilized on a solid support under conditions allowing the formation of a complex between the ligand and the enzyme.

The ligand may be any chemical molecule being able to bind to the given protein, preferably the given enzyme. This includes natural ligands of the protein including proteins.

Preferably, said ligand is selected from the group consisting of synthetic or naturally occurring chemical compounds or organic synthetic drugs, more preferably small molecules, organic drugs or natural small molecule compounds. Such small molecules are preferably not proteins or nucleic acids. Preferably, small molecules exhibit a molecular weight of less than 1000 Da, more preferred less than 750 Da, most preferred less than 500 Da.

In a preferred embodiment, the ligand is a broad specificity (non-selective) ligand that can bind to multiple members of the enzyme family of interest.

In an alternatively preferred embodiment, the ligand is an antibody.

In the context of the present invention, the term “antibody” refers to any kind of immunoglobulin-derived structure with binding specificity to a protein of interest, preferably with binding specificity to an enzyme of interest, including, but not limited to, a full length antibody, an antibody fragment (a fragment derived, physically or conceptually, from an antibody structure), a derivative of any of the foregoing, a chimeric molecule, a fusion of any of the foregoing with another polypeptide, or any alternative structure/composition. An antibody of the invention may be any polypeptide which comprises at least one antigen binding fragment. Antigen binding fragments consist of at least the variable domain of the heavy chain and the variable domain of the light chain, arranged in a manner that both domains together are able to bind to the specific antigen. An antibody fragment contains at least one antigen binding fragment as defined above, and exhibits essentially the same function and specificity as the complete antibody of which the fragment is derived from.

Preferably, the antibody of the invention is selected from the group consisting of monoclonal antibodies, polyclonal antibodies, chimeric antibodies, and antibody fragments.

Monoclonal antibodies are monospecific antibodies that are identical because they are produced by one type of immune cell that are all clones of a single parent cell. Polyclonal antibodies include, for example, antibodies derived from a patient suffering from an autoimmune disease. A chimeric antibody is an antibody in which at least one region of an immunoglobulin of one species is fused to another region of an immunoglobulin of another species by genetic engineering in order to reduce its immunogenicity. For example, murine V_{L} and V_{H} regions may be fused to the remaining part of a human immunoglobulin. In addition, the antibody of the present invention may be an antibody fragment in form of an antibody domain (Fab), a single chain antibody, or a biological receptor or receptor fusion protein (Chames et al., 2009. Br. J. Pharmacol. 157(2): 220-233).

Preferably, the ligand binds to the active site (catalytic site) of the enzyme of interest.

Equally preferred, the ligand binds to a site that is different from the active site, for example to an allosteric site of the enzyme of interest.

According to the invention, it is possible to use one type of ligand. However, it is equally possible to use more than one type of ligands, e.g. up to 10 different ligands.

Furthermore, according to the invention, also a protein-interacting compound is added to the protein preparations in order to determine the species profile of said compound. The concept underlying the invention is to determine whether the compound interferes with the formation of the complex between the ligand and the protein, preferably the enzyme. According to the invention, also the compound may be any chemical molecule being able to bind to the given protein, preferably the given enzyme. This includes natural ligands of the protein including proteins. Preferably, also the compound is selected from the group consisting of synthetic or naturally occurring chemical compounds or organic synthetic drugs, more preferably small molecules, organic drugs or natural small molecule compounds. Such small molecules are preferably not proteins or nucleic acids. Preferably, small molecules exhibit a molecular weight of less than 1000 Da, more preferred less than 750 Da, most preferred less than 500 Da.

In an alternatively preferred embodiment, the compound is an antibody.

In a further preferred embodiment, the compound and the ligand of the invention are both antibodies.

In a preferred embodiment of the present invention, the compound has already been tested for its capacity to be a protein-interacting compound, with the consequence that in the method of the invention, the species profile of a compound known to be a protein-interacting compound, preferably an enzyme-interacting compound is determined.

However, it is also included within the present invention that the compound has not been tested before for its capacity to be a protein-interacting compound. In this case, the invention preferably also relates to a method for the identification of a protein-interacting compound, preferably an enzyme-interacting compound. Preferably, said compound is identified starting from a library containing such compounds, preferably small molecules. Then, in the course of the present invention, such a library is screened.

A “library” according to the present invention relates to a (mostly large) collection of (numerous) different chemical entities that are provided in a sorted manner that enables both a fast functional analysis (screening) of the different individual entities, and at the same time provide for a rapid identification of the individual entities that form the library. Examples are collections of tubes or wells or spots on surfaces that contain chemical compounds that can be added into reactions with one or more defined potentially interacting partners in a high-throughput fashion. After the identification of a desired “positive” interaction of both partners, the respective compound can be rapidly identified due to the library construction. Libraries of synthetic and natural origins can either be purchased or designed by the skilled artisan.

In the context of the present invention, the term “interacting” means that the compound interferes in some way with the protein, preferably the enzyme.

Preferably, the compound binds directly or indirectly to the protein and, thereby, interferes with the binding to the ligand. The compound may bind to the active site of the protein, preferably the enzyme or to an allosteric site. Preferably, the compound modulates the activity of the protein. More preferably, the compound is an inhibitor of the activity of the protein, preferably the enzyme, but it may also be an activator of the activity of the protein, preferably the enzyme.

In the present invention, the term “complex” denotes a complex where the ligand interacts with the protein, preferably the enzyme, e.g. by covalent or, most preferred, by non-covalent binding.

The skilled person will know which conditions can be applied in order to enable the formation of said complex.

In the context of the present invention, the term “under conditions allowing the formation of the complex” includes all conditions under which such formation, preferably such binding is possible. This includes the possibility of having the solid support on an immobilized phase and pouring the lysate onto it. In another preferred embodiment, it is also included that the solid support is in a particulate form and mixed with the cell lysate.

In the context of non-covalent binding, the binding between the ligand and the protein is, e.g., via salt bridges, hydrogen bonds, hydrophobic interactions or a combination thereof.

In a preferred embodiment, the steps of the formation of the complex are performed under essentially physiological conditions. The physical state of proteins within cells is described in Petty, 1998 (Howard R. Petty, Chapter 1, Unit 1.5 in: Juan S. Bonifacino, Mary Dasso, Joe B. Harford, Jennifer Lippincott-Schwartz, and Kenneth M. Yamada (eds.) Current Protocols in Cell Biology Copyright © 2003 John Wiley & Sons, Inc. All rights reserved. DOI: 10.1002/0471143030.cb0101s00 Online Posting Date: May, 2001 Print Publication Date: October, 1998).

The contacting under essentially physiological conditions has the advantage that the interactions between the ligand, the cell preparation (i. e. the kinase to be characterized) and optionally the compound reflect as much as possible the natural conditions. “Essentially physiological conditions” are inter alia those conditions which are present in the original, unprocessed sample material. They include the physiological protein concentration, pH, salt concentration, buffer capacity and post-translational modifications of the proteins involved. The term “essentially physiological conditions” does not require conditions identical to those in the original living organism, wherefrom the sample is derived, but essentially cell-like conditions or conditions close to cellular conditions. The person skilled in the art will, of course, realize that certain constraints may arise due to the experimental set-up which will eventually lead to less cell-like conditions. For example, the eventually necessary disruption of cell walls or cell membranes when taking and processing a sample from a living organism may require conditions which are not identical to the physiological conditions found in the organism. Suitable variations of physiological conditions for practicing the methods of the invention will be apparent to those skilled in the art and are encompassed by the term “essentially physiological conditions” as used herein. In summary, it is to be understood that the term “essentially physiological conditions” relates to conditions close to physiological conditions, as e. g. found in natural cells, but does not necessarily require that these conditions are identical.

For example, “essentially physiological conditions” may comprise 50-200 mM NaCl or KCl, pH 6.5-8.5, 20-37° C., and 0.001-10 mM divalent cation (e.g. Mg++, Ca++,); more preferably about 150 m NaCl or KCl, pH7.2 to 7.6, 5 mM divalent cation and often include 0.01-1.0 percent non-specific protein (e.g. BSA). A non-ionic detergent (Tween®, NP-40, Triton® X-100) can often be present, usually at about 0.001 to 2%, typically 0.05-0.2% (volume/volume). For general guidance, the following buffered aequous conditions may be applicable: 10-250 mM NaCl, 5-50 mM Tris HCl, pH5-8, with optional addition of divalent cation(s) and/or metal chelators and/or non-ionic detergents.

Preferably, “essentially physiological conditions” mean a pH of from 6.5 to 7.5, preferably from 7.0 to 7.5, and/or a buffer concentration of from 10 to 50 mM, preferably from 25 to 50 mM, and/or a concentration of monovalent salts (e.g. Na or K) of from 120 to 170 mM, preferably 150 mM. Divalent salts (e.g. Mg or Ca) may further be present at a concentration of from 1 to 5 mM, preferably 1 to 2 mM, wherein more preferably the buffer is selected from the group consisting of Tris-HCl or HEPES.

In a preferred embodiment of the present invention, the ligand may be immobilized on a solid support. Throughout the invention, the term “solid support” relates to every undissolved support being able to immobilize a small molecule ligand on its surface.

According to a further preferred embodiment, the solid support is selected from the group consisting of agarose, modified agarose, crosslinked polysaccharide polymer materials, such as sepharose® beads, including, e.g., NHS-activated sepharose® (GE Health Care), latex, cellulose, and ferro- or ferrimagnetic particles.

The ligand may be coupled to the solid support either covalently or non-covalently. Non-covalent binding includes binding via biotin affinity ligands binding to streptavidin matrices. Antibodies may be coupled non-covalently to protein A or protein G containing supports.

Preferably, the ligand is covalently coupled to the solid support.

Before the coupling, the matrixes can contain active groups such as NHS, Carbodiimide etc. to enable the coupling reaction with the ligand. The ligand can be coupled to the solid support by direct coupling (e.g. using functional groups such as amino-, sulfhydryl-, carboxyl-, hydroxyl-, aldehyde-, and ketone groups) and by indirect coupling (e.g. via biotin, biotin being covalently attached to the ligand and non-covalent binding of biotin to streptavidin which is bound to solid support directly).

The linkage to the solid support material may involve cleavable and non-cleavable linkers. The cleavage may be achieved by enzymatic cleavage or treatment with suitable chemical methods.

Preferred binding interfaces for binding the ligand to solid support material are linkers with a C-atom backbone. Typically linkers have a backbone of 8, 9 or 10 atoms. The linkers contain either a carboxy or amino functional group, preferably a primary amino group.

In a preferred embodiment of the invention, the protein-containing protein preparation (irrespective which species it is derived from) is first incubated with the compound and then with the ligand. However, the simultaneous incubation of the compound and the ligand (coincubation) with the protein-containing protein preparation is equally preferred (competitive binding assay).

In case that the incubation with the compound is first, the protein, preferably the enzyme, is preferably first incubated with the compound for 10 to 60 minutes, more preferred for 30 to 45 minutes at a temperature of 4° C. to 37° C., more preferred at 4° C. to 25° C., most preferred at 4° C. Preferably compounds are used at concentrations ranging from 1 nM to 1 mM, preferably from 1 nM to 100 μM, preferably from 1 nM to 10μM. The second step, contacting with the immobilized ligand, is preferably performed for 10 to 60 minutes at 4° C.

In case of simultaneous incubation, the protein, preferably the enzyme, is preferably simultaneously incubated with the compound and ligand for 30 to 120 minutes, more preferred for 60 to 120 minutes at a temperature of 4° C. to 37° C., more preferred at 4° C. to 25° C., most preferred at 4° C. Preferably compounds are used at concentrations ranging from 1 nM to 1 mM, preferably from 1 nM to 100 μM, preferably from 1 nM to 10 μM.

The skilled person will appreciate that between the individual steps of the methods of the invention, washing steps may be necessary. Such washing is part of the knowledge of the person skilled in the art. The washing serves to remove non-bound components of the cell lysate from the solid support. Nonspecific (e.g. simple ionic) binding interactions can be minimized by adding low levels of detergent or by moderate adjustments to salt concentrations in the wash buffer.

According to the invention, the complexes formed in step d) are detected with the help of mass spectrometry.

In principle, there are several procedures for detecting the complexes. This includes that the protein, preferably the enzyme, is separated from the ligand and is then detected by mass spectrometry (MS) or it includes that the protein, preferably the enzyme, is separated from the ligand and its amount is then detected by mass spectrometry. However, according to the invention, also methods are included wherein the complexes are detected without separating the protein, preferably the enzyme, from the ligand.

According to the invention, it is equally possible to replace the detection by mass spectrometry by any other method capable of detecting proteins including the detection with the help of protein-specific antibodies. In this case, the separating step may not be necessary, because species selective antibodies could be used. These are antibodies that selectively bind to the species-1 variant or species-2 variant of the protein, preferably the enzyme.

In one embodiment of the method according to the invention, the complex formed during the method of the invention is detected. The fact that such complex is formed preferably indicates that the compound does not completely inhibit the formation of the complex. On the other hand, if no complex is formed, the compound is presumably a strong interact or of the protein, which is indicative for its therapeutic potential.

According to another embodiment of the invention, the amount of the complex formed during the method is determined. In general, the less complex in the presence of the respective compound is formed, the stronger the respective compound interacts with the protein, which is indicative for its therapeutic potential.

According to invention, separating means every action which destroys the interactions between the ligand and the protein, preferably the enzyme. This includes in a preferred embodiment the elution of the ligand from the protein, preferably the enzyme.

The elution can be achieved by using non-specific reagents as described in detail below (ionic strength, pH value, detergents).

Such non-specific methods for destroying the interaction are principally known in the art and depend on the nature of the ligand-protein interaction. Principally, change of ionic strength, the pH value, the temperature or incubation with detergents are suitable methods to dissociate the target protein from the immobilized ligand. The application of an elution buffer can dissociate binding partners by extremes of pH value (high or low pH; e.g. lowering pH by using 0.1 M citrate, pH2-3), change of ionic strength (e.g. high salt concentration using NaI, KI, MgCl₂, or KCl), polarity reducing agents which disrupt hydrophobic interactions (e.g. dioxane or ethylene glycol), or denaturing agents (chaotropic salts or detergents such as Sodium-docedyl-sulfate, SDS; Review: Subramanian A., 2002, Immunoaffinity chromatography).

In some cases, the solid support has preferably to be separated from the released material. The individual methods for this depend on the nature of the solid support and are known in the art. If the support material is contained within a column the released material can be collected as column flowthrough. In case the support material is mixed with the lysate components (so called batch procedure) an additional separation step such as gentle centrifugation may be necessary and the released material is collected as supernatant. Alternatively magnetic beads can be used as solid support so that the beads can be eliminated from the sample by using a magnetic device.

The identification of proteins with mass spectrometric analysis (mass spectrometry) is known in the art (Shevchenko et al., 1996, Analytical Chemistry 68: 850-858; Mann et al., 2001, Analysis of proteins and proteomes by mass spectrometry, Annual Review of Biochemistry 70, 437-473) and is further illustrated in the example section.

Preferably, the mass spectrometry analysis is performed in a quantitative manner, for example by using iTRAQ® technology (isobaric tags for relative and absolute quantification) or cICAT® (cleavable isotope-coded affinity tags) (Wu et al., 2006. J. Proteome Res. 5, 651-658). Alternatively, tandem mass tag (TMT®) reagents can be used (commercially available from, e.g., Thermo Scientific). The tandem mass tag (TMT®) reagents are a set of multiplexed, amine-specific, stable isotope reagents that can label peptides in up to six different biological samples enabling simultaneous identification and quantitation of peptides.

Accordingly, in a preferred embodiment, detecting the complexes and/or determining the amount of the complexes formed in step e) is performed with the help of quantitative mass spectrometry.

The advantage of using quantitative mass spectrometry in the context of the present invention is that relative amounts of the differently formed complexes and/or different species specific peptides can be compared in one set of experiment.

According to a further preferred embodiment of the present invention, the characterization by mass spectrometry (MS) is performed by the identification of species-specific peptides of the protein, preferably the enzyme. Therefore, a species-specific peptide is used in the present invention as an experimentally well observable peptide that uniquely identifies a protein ortholog.

Bioinformatic methods for the identification of unique species-specific peptides are known in the art, for example by creating sequence alignments (Chenna et al, 2003. Nucleic Acids Res. 31(13):3497-500) as illustrated in FIGS. 5 and 7.

According to a preferred embodiment, the characterization is performed by quantifying the species-specific peptides obtained in the course of practicing the methods of the invention.

Therefore, in a preferred embodiment, the method of the present invention includes the quantitation of species-specific peptides.

Preferably, quantitation of species-specific peptides is performed with the help of quantitative mass spectrometry. The quantiation of species-specific peptides by means of quantitative mass spectrometry is further illustrated in the example section of the present invention.

It is one purpose of the present invention to detect the species profile of a given compound. Therefore dose-response curves (IC₅₀ values) are calculated for each protein ortholog and the ratio of IC₅₀ values is compared. Furthermore, K_{D} values can be determined and compared.

Preferably, a difference between the amount of complexes formed between the species-1-protein variant and the ligand and the species-2-protein variant and the ligand indicates that the compound has a higher affinity for the species-variant which forms less complexes.

Preferably, the identification methods of the invention are performed as a medium or high throughput screening.

In a preferred embodiment of the present invention, said species-1 is human.

Furthermore, in another preferred embodiment of the invention, said species-2 is a rodent, preferably rat or mouse.

Equally preferred species are rabbit, dog, pig, and monkeys such as Macaca fasciculari.

The present invention is further defined by the attached figures and examples, which are intended to illustrate, but not to limit the scope of the present invention.

### BRIEF DESCRIPTION OF THE FIGURES

FIG. 1: Structure of compound 1.

FIG. 2: Dose response curves of compound 1 for human and mouse lipid kinases. The experiment was performed as described in Example 1 with a mix of a human cell lysate (HL60) and a mouse cell lysate (Raw 264.7). (A) Human PI3Kbeta (hPIK3CB). (B) Mouse PI3Kbeta (mPIK3CB).

FIG. 3: Dose response curves of compound 1 for human and mouse lipid kinases. The experiment was performed as described in Example 1 with a mix of a human cell lysate (HL60) and a mouse cell lysate (Raw 264.7). (A) Human PI3Kgamma (hPIK3CG). (B) Mouse PI3Kgamma (mPIK3CG).

FIG. 4: Dose response curves of compound 1 for human and mouse lipid kinases. The experiment was performed as described in Example 1 with a mix of a human cell lysate (HL60) and a mouse cell lysate (Raw 264.7). (A) Human PI3Kdelta (hPIK3CD). (B) Mouse PI3Kdelta (mPIK3CD).

FIG. 5: Extract of a Clustal W-based multiple alignment of the first 360 residues of human and mouse PIK3gamma protein sequences. Tryptic peptide sequences unique to the homolog of each species are depicted as dashed or solid lines below the alignment. The bottom-most line represents the standard Clustal-W consesus: ‘*’ signifies that the residues in that column are identical in all sequences in the alignment. ‘:’ means that conserved substitutions have been observed and ‘.’ denotes semi-conserved substitutions.

FIG. 6: Structure of compound 2.

FIG. 7: Clustal W-based multiple alignment of human, mouse and rat PIK3gamma (PIK3CG) protein sequences. Tryptic peptide sequences unique to each species are depicted below the alignment (=== mouse specific peptides, _{———} rat specific peptides, ——— human specific peptides). The bottom line represents the standard Clustal-W consensus sequence: ‘*’ signifies identical residues in all sequences of the alignment. ‘:’ means conserved substitutions and ‘.’ denotes semi-conserved substitutions.

FIG. 8: Species profile of compound 2 using extract mixtures of human (Jurkat) and rodent cell lines (rat RBL-1 and mouse RAW264.7 cells).

EXAMPLES

### Example 1: Species Profile of Compound 1

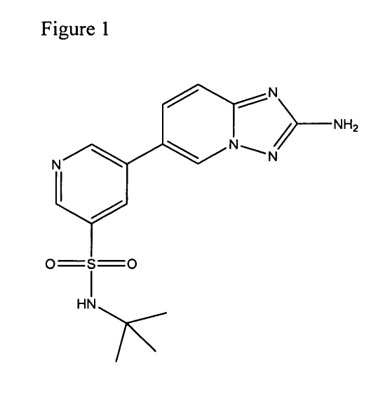

This example demonstrates the species profile of the kinase inhibitor compound 1 (FIG. 1) for human and mouse lipid kinases. The synthesis of compound 1 has previously been described (WO 2009/068482).

Principle of the Assay

Cell lysates from a human (HL60) and a mouse cell line (Raw 264.7) were prepared and subsequently mixed at a 1:1 ratio. Compound 1 was added at various concentrations (3 μM, 0.75 μM, 0.047 μM, 0.012 μM, plus DMSO solvent control) to aliquots of this mixed cell lysate and allowed to bind to the proteins in the lysate. Subsequently, the affinity matrix (beads with immobilized ligands) was added to each aliquot to capture proteins that were not interacting with compound 1. The generation of the affinity matrix (beads with immobilized ligands) was described in WO 2009/098021.

After the incubation time of 60 minutes the beads with captured proteins were separated from the lysate and bead bound proteins were eluted in SDS sample buffer and subsequently separated by SDS-Polyacrylamide gel electrophoresis. Electrophoresis was stopped after 20 minutes and the gel was stained with colloidal Coomassie. The stained area of each gel lane was cut out and subjected to in-gel proteolytic digestion with trypsin. Peptides originating from the different aliquots treated with compound 1 and the DMSO solvent control were labeled with isobaric tagging reagents (Tandem mass tag (TMT®) reagents, Thermofisher). The TMT® reagents are a set of multiplexed, amine-specific, stable isotope reagents that can label peptides in up to six different biological samples enabling simultaneous identification and quantitation of peptides. The combined samples were analyzed with a nano-flow liquid chromatography system coupled online to a tandem mass spectrometer (LC-MS/MS) experiment followed by reporter ion quantitation in the MS/MS spectra (Ross et al., 2004. Mol. Cell. Proteomics 3(12):1154-1169; Dayon et al., 2008. Anal. Chem. 80(8):2921-2931; Thompson et al., 2003. Anal. Chem. 75(8):1895-1904). Further experimental protocols can be found in WO2006/134056 and a previous publication (Bantscheff et al., 2007. Nature Biotechnology 25, 1035-1044).

Results

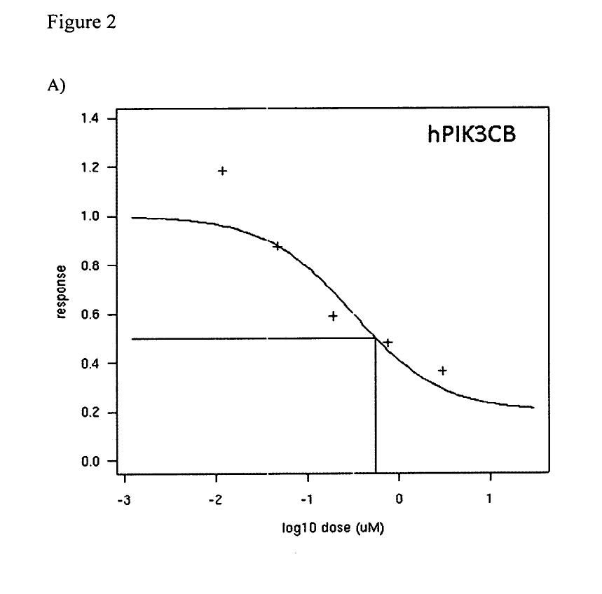

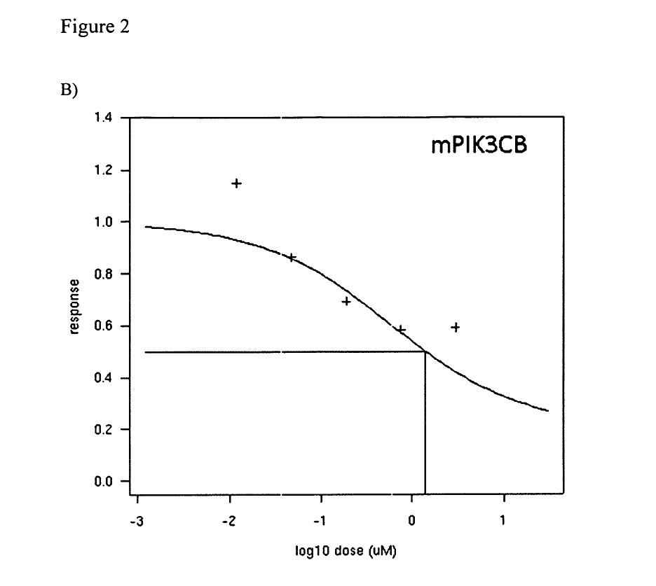

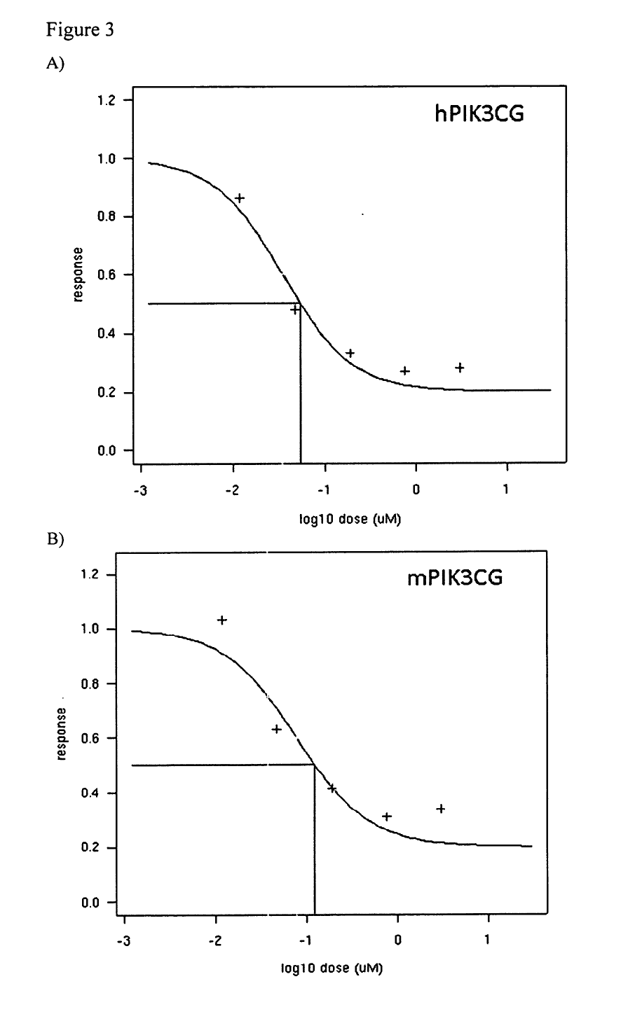

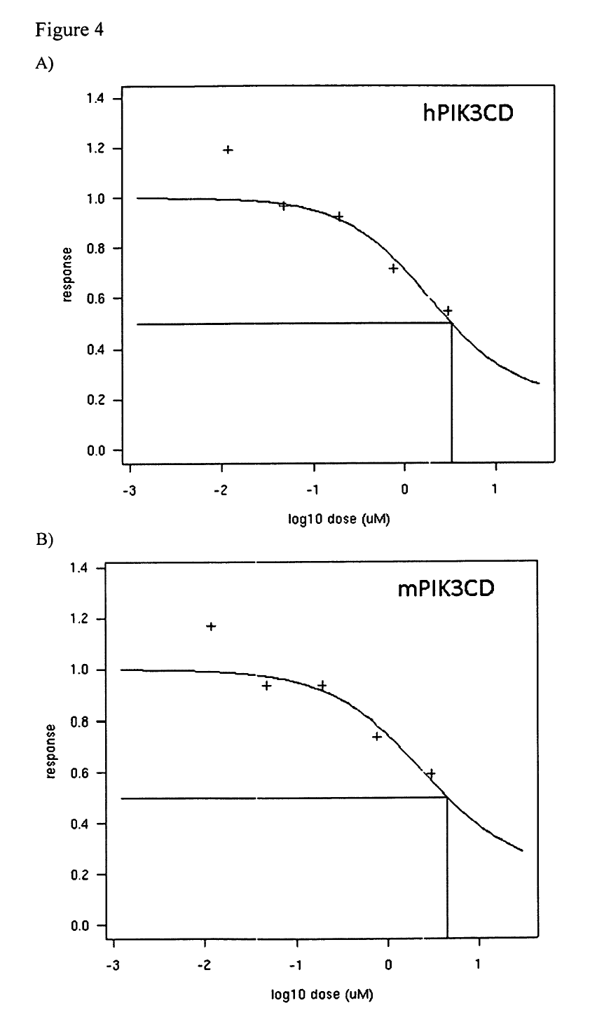

Table 5 shows the IC₅₀ inhibition values of compound 1 for individual human and mouse lipid kinase homologues. Dose response curves are shown in FIGS. 2, 3 and 4. The result shows that the ratio of the IC₅₀ values for the human and mouse PIK3CG (PI3Kγ) protein is 2.61 (potency ratio across species). Thus compound 1 is significantly more potent for the human form of PIK3CG (PI3Kγ) compared to the mouse PIK3CG (PI3Kγ). The potency ratio for PIK3CD is 1.0 showing that compound 1 is equipotent for the human and mouse forms of PIK3CD (PI3Kδ).

Comparison of the IC₅₀ values for two different kinases within one species gives the selectivity window. For example, in humans the ratio of the IC₅₀ for PIK3CD and PIK3CG is 48, showing that compound 1 is 48-fold more potent for PIK3CG compared to PIK3CD. In mouse, the IC₅₀ ration for PIK3CD and PIK3CG is only 23.

Protocols

1. Cell Culture

HL60 cells (DSMZ #ACC3) were either obtained from an external supplier (CIL SA, Mons, Belgium) or grown in one litre Spinner flasks (Integra Biosciences, #182101) in suspension in RPMI 1640 medium (Invitrogen, #21875-034) supplemented with 20% Fetal Bovine Serum (Invitrogen, #10270-106). Cells were harvested by centrifugation, washed once with 1× PBS buffer (Invitrogen, #14190-094) and cell pellets were frozen in liquid nitrogen and subsequently stored at −80° C.

RAW264.7 cells (ATCC, #TIB-71) were grown adherent in 15cm plates (BD, #353025) in DMEM medium (Invitrogen, #41965) supplemented with 10% heat inactivated Fetal Bovine Serum (Invitrogen, #10270-106), and 1.5 g/L sodium bicarbonate (Invitrogen, #25080). Cells were harvested by cell scraper (BD, #353085), washed with 1× PBS buffer (Invitrogen, #14190-094) and cell pellets were frozen in liquid nitrogen and subsequently stored at −80° C.

2. Preparation of Cell Lysates

Cells were homogenized in a Potter S homogenizer in lysis buffer: 50 mM Tris-HCl, 0.8% NP40, 5% glycerol, 150 mM NaCl, 1.5 mM MgCl₂, 25 mM NaF, 1 mM sodium vanadate, 1 mM DTT, pH 7.5. One complete EDTA-free tablet (protease inhibitor cocktail, Roche Diagnostics, 1 873 580) per 25 ml buffer was added. The material was dounced 20 times using a mechanized POTTER S, transferred to 50 ml falcon tubes, incubated for 30 minutes rotating at 4° C. and spun down for 10 minutes at 20,000×g at 4° C. (10,000 rpm in Sorvall SLA600, precooled). The supernatant was transferred to an ultracentrifuge (UZ)-polycarbonate tube (Beckmann, 355654) and spun for 1 hour at 145.000×g at 4° C. (40.000 rpm in Ti50.2, pre-cooled). The supernatant was transferred again to a fresh 50 ml falcon tube, the protein concentration was determined by a Bradford assay (BioRad) and samples containing 5 mg of protein per aliquot were prepared. The samples were immediately used for experiments or frozen in liquid nitrogen and stored frozen at −80° C. Cell extracts from human HL60 cells and mouse raw 264.7 cells were mixed in a one to one (1:1) ratio.

3. Capturing of Proteins from Cell Lysate

Sepharose®-beads with the immobilized ligands (35 μl beads per aliquot) were equilibrated in lysis buffer and incubated with a cell lysate sample containing 5 mg of protein on an end-over-end shaker (Roto Shake Genie, Scientific Industries Inc.) for 60 minutes at 4° C. Beads were collected, transferred to Mobicol-columns (MoBiTech 10055) and washed with 10 ml lysis buffer containing 0.4% NP40 detergent, followed by 5 ml lysis buffer containing 0.2% detergent. To elute bound proteins, 60 μl 2× SDS sample buffer was added to the column. The column was incubated for 30 minutes at 4° C. and the eluate was transferred to a siliconized microfuge tube by centrifugation. Proteins were then alkylated with 108 mM iodoacetamid. Proteins were then separated by SDS-Polyacrylamide electrophoresis (SDS-PAGE). Suitable gel bands were cut out and subjected to in-gel proteolytic digestion with trypsin.

4. Protein Identification and Quantitation by Mass Spectrometry

4.1 Protein Digestion Prior to Mass Spectrometric Analysis

Gel-separated proteins were digested in-gel essentially following a previously described procedure (Shevchenko et al., 1996, Anal. Chem. 68: 850-858). Briefly, gel-separated proteins were excised from the gel using a clean scalpel, destained twice using 100 μl 5 mM triethylammonium bicarbonate buffer (TEAB; Sigma T7408) and 40% ethanol in water and dehydrated with absolute ethanol. Proteins were subsequently digested in-gel with porcine trypsin (Promega) at a protease concentration of 10 ng/μl in 5 mM TEAB. Digestion was allowed to proceed for 4 hours at 37° C. and the reaction was subsequently stopped using 5 μl 5% formic acid.

4.2 Sample Preparation Prior to Analysis by Mass Spectrometry

Gel plugs were extracted twice with 20 μl 1% formic acid and three times with increasing concentrations of acetonitrile. Peptide extracts were subsequently pooled with acidified digest supernatants and dried in a vacuum centrifuge.

4.3 Tandem Mass Tag (TMT®) Labeling of Peptide Extracts

The peptide extracts corresponding to the different aliqots treated with different concentrations of compound 1 were labeled with variants of the isobaric tagging reagent as shown in Table 1 (TMT® sixplex Label Reagent Set, part number 90066, Thermo Fisher Scientific Inc., Rockford, Ill. 61105 USA). The tandem mass tag (TMT®) reagents are a set of multiplexed, amine-specific, stable isotope reagents that can label peptides on amino groups in up to six different biological samples enabling simultaneous identification and quantitation of peptides. The TMT® reagents were used according to instructions provided by the manufacturer. The samples were resuspended in 10 μl 50 mM TEAB solution, pH 8.5 and 10 pl acetonitrile were added. The TMT® reagent was dissolved in acetonitrile to a final concentration of 24 mM and 10 μl of reagent solution were added to the sample. The labeling reaction was performed at room temperature for one hour on a horizontal shaker and stopped by adding 5 μl of 100 mM TEAB and 100 mM glycine in water. The labeled samples were then combined, dried in a vacuum centrifuge and resuspended in 60% 200 mM TEAB/40% acetonitrile. 2 μl of a 2.5% NH₂OH solution in water were added, incubated for 15 min and finally the reaction was stopped by addition of 10 μl of 20% formic acid in water. After freeze-drying samples were resuspended in 50 μl 0.1% formic acid in water.

TABLE 1
Labeling of peptides with TMT ® isobaric tagging reagents

| | Concentration | |
|---|---|---|
| Gel lane | of compound 1 (μM) | TMT6 reagent |
| 1 | 3.0 | 126 |
| 2 | 0.75 | 127 |
| 3 | 0.188 | 128 |
| 3 | 0.047 | 129 |
| 5 | 0.012 | 130 |
| 6 | 0 | 131 |

4.4 Creation of Mixed Species Inclusion Lists

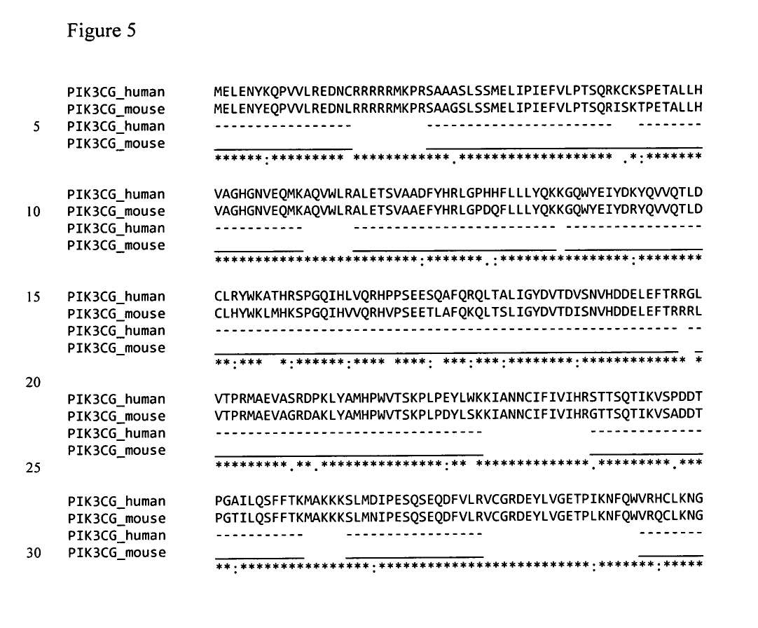

Tryptic peptides that are only present in mouse or in human proteomes were identified for the proteins of interest, i.e. unique for mouse or for human version of the protein, (FIG. 5). Subsequently for each human and mouse protein that was of interest to the study the selected peptides were ranked according to the quality of the quantification and ease of identification inherent to these peptides, additionally there retention times were aligned in order to make the detection as sensitive and specific as possible, (Savitski et al., 2010. Targeted Data Acquisition for Improved Reproducibility and Robustness of Proteomic Mass Spectrometry Assays. J. Am. Soc. Mass. Spectrom. 21(10):1668-1679). All possible unique peptides for each protein were selected, even those that had not been detected in mass spectrometric experiments before and their mass over charge values together with retention time windows were fed into the instrument software.

FIG. 5 displays aligned human and mouse PI3Kgamma protein sequences with differing peptides marked. Table 2 and table 3 show an example of 10 peptides that are used for the inclusion list for PIK3CG for human (table 1) and mouse (table 2) sequences. All peptides are TMT6plex® modified on the lysines and the N-terminus, the cysteines are carbamidomethylated.

TABLE 2
Human PI3K gamma, IPI00292690.1

| | | norm | |
|---|---|---|---|
| Sequence | m/z | RT | Charge |
| STTSQTIK | 662.397 | 0.31 | 2 |
| GEDQGSFNADK | 813.412 | 0.34 | 2 |
| AGALAIEK | 615.894 | 0.49 | 2 |
| GALLNLQIYCGK | 904.528 | 0.68 | 2 |
| QKLENLQNSQLPESFR | 797.112 | 0.54 | 3 |
| IQQSTVGNTGAFKDEVLNHWLK | 793.948 | 0.70 | 4 |
| VCGRDEYLVGETPIK | 732.069 | 0.53 | 3 |
| PLPEYLWK | 752.452 | 0.72 | 2 |
| VSPDDTPGAILQSFFTK | 761.087 | 0.88 | 3 |
| VSPDDTPGAILQSFFTK | 1141.127 | 0.88 | 2 |

TABLE 3
Mouse PI3K gamma, IPI00468012.1

| | | norm | |
|---|---|---|---|
| Sequence | m/z | RT | Charge |
| ASAETPGSESK | 761.411 | 0.28 | 2 |
| SEEDAK | 568.813 | 0.25 | 2 |
| AGTLVIEK | 644.915 | 0.53 | 2 |
| AEEQGSFNADK | 827.427 | 0.35 | 2 |
| QKLESLQNSNLPESFR | 783.437 | 0.55 | 3 |
| LGPDQFLLLYQK | 631.713 | 0.79 | 3 |
| GALLNLQIYCCK | 956.033 | 0.69 | 2 |
| HVPSEETLAFQK | 615.348 | 0.49 | 3 |
| YQVVQTLDCLHYWK | 771.082 | 0.71 | 3 |
| VTIDIK | 573.879 | 0.56 | 2 |

4.5 Mass Spectrometric Data Acquisition

Peptide samples were injected into a nano LC system (CapLC, Waters or nano-LC 1D+, Eksigent) which was directly coupled either to a quadrupole TOF (QTOF Ultima, QTOF Micro, Waters), ion trap (LTQ) or Orbitrap mass spectrometer. Peptides were separated on the LC system using a gradient of aqueous and organic solvents (see below). Solvent A was 0.1% formic acid and solvent B was 70% acetonitrile in 0.1% formic acid.

TABLE 4
Peptide elution off the LC system

| | Flow rate | Gradient Time |
|---|---|---|
| Method file | (nL/min) | (min)-% B |
| HCD_265min | 190 | 00-5.263 |
| | | 07-10 |
| | | 190-40.263 |
| | | 210-52.105 |
| | | 223-60 |
| | | 230-90 |
| | | 236-90 |
| | | 240-5.263 |
| | | 260-5.263 |

4.6 Protein Identification

The peptide mass and fragmentation data generated in the LC-MS/MS experiments were used to query a protein data base consisting of an in-house curated version of the International Protein Index (IPI) protein sequence database combined with a decoy version of this database (Elias and Gygi, 2007, Target-decoy search strategy for increased confidence in large-scale protein identifications by mass spectrometry. Nature Methods 4, 207-214). Proteins were identified by correlating the measured peptide mass and fragmentation data with data computed from the entries in the database using the software tool Mascot (Matrix Science)(Perkins et al., 1999. Probability-based protein identification by searching sequence databases using mass spectrometry data. Electrophoresis 20, 3551-3567). Search criteria varied depending on which mass spectrometer was used for the analysis. Protein acceptance thresholds were adjusted to achieve a false discovery rate of below 1% as suggested by hit rates on the decoy data base (Elias and Gygi, 2007, Target-decoy search strategy for increased confidence in large-scale protein identifications by mass spectrometry. Nature Methods 4, 207-214).

4.7 Use of Species-Specific Peptides for the Protein Quantitation

Relative protein quantitation was performed using peak areas of iTMT® reporter ion signals essentially as described in an earlier publication (Bantscheff et al., 2007. Nature Biotechnology 25, 1035-1044). Quantitation of the mouse and human proteins was performed using peptides that were unique to one of the species. Centroided TMT® ®reporter ion signals were computed by the XCalibur software and extracted from MS data files using in house scripts. Reporter ion intensities were multiplied with the ion accumulation time yielding an area value proportional to the number of reporter ions present in the mass analyzer. Fold changes are reported based on reporter ion areas in comparison to the untreated sample and are calculated using sum-based bootstrap algorithm. Fold changes were corrected for isotope purity and adjusted for interference caused by co-eluting nearly isobaric peaks as estimated by the s2i measure (Savitski et al., 2010. Targeted Data Acquisition for Improved Reproducibility and Robustness of Proteomic Mass Spectrometry Assays. J. Am. Soc Mass. Spectrom. 21(10): 1668-79).

4.8 Determination of IC₅₀ Values Based on Dpecies Specific Unique Peptides

Dose-response curves were fitted using R (www.r-project.org) (R Development Core Team R: A language and environment for statistical computing. Vienna, Austria, 2007) and the drc package (www.bioassay.dk) (Ritz, C. & Streibig, J. C. Bioassay Analysis using R. J. Statist. Software 12, (2007) as described previously (Bantscheff et al., 2007, Nat. Biotechnol. 25, 1035-1044).

TABLE 5
Human and mouse species profiles for compound 1.
Lipid kinase binding data (IC₅₀ values) of compound 1 is shown for two replicate profiling
experiments using human HL-60 cells and mouse RAW 264.7 cells. Average ratios were
calculated by dividing potencies across the species and averaging the ratios.

| | Experiment 1 (human/mouse 1) | | | | Experiment 2 (human/mouse 2) | | | | Average |
|---|---|---|---|---|---|---|---|---|---|
| | Number of | | | | Number of | | | | ratio |
| | unique spectra | | IC₅₀ | | unique spectra | | IC₅₀ | | human |
| Kinase | human | mouse | human | mouse | human | mouse | human | mouse | mouse |
| PIK3C3 | 4 | 2 | 3 | 3 | n.d. | n.d. | n.d. | n.d. | n.d. |
| PIK3Ca | 13 | 7 | 2.372 | 3 | 12 | 9 | 2.599 | 3 | app. 1 |
| PIK3Cb | 5 | 25 | 0.136 | 0.251 | 7 | 26 | 0.283 | 0.162 | 0.87 |
| PIK3Cd | 177 | 136 | 1.061 | 1.314 | 79 | 63 | 1.525 | 1.288 | 1.00 |
| PIK3Cg | 74 | 91 | 0.022 | 0.057 | 26 | 52 | 0.027 | 0.071 | 2.61 |
| PIK3R4 | 8 | n.d. | 3 | n.d. | n.d. | n.d. | n.d. | n.d. | n.d. |
| PIK4Ca | 16 | 5 | 3 | 3 | 7 | 16 | 3 | 3 | n.d. |
| PIK4Cb | 11 | 20 | 3 | 3 | 4 | 14 | 3 | 3 | n.d. |

Example 2: Species Profile of Compound 2

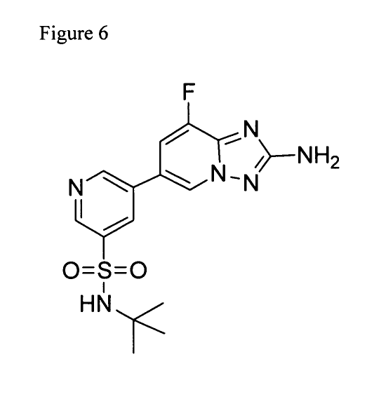

This example demonstrates the species profile of the kinase inhibitor compound 2 (FIG. 6) for human and rodent lipid kinases. The synthesis of compound 2 has previously been described (WO 2009/068482).

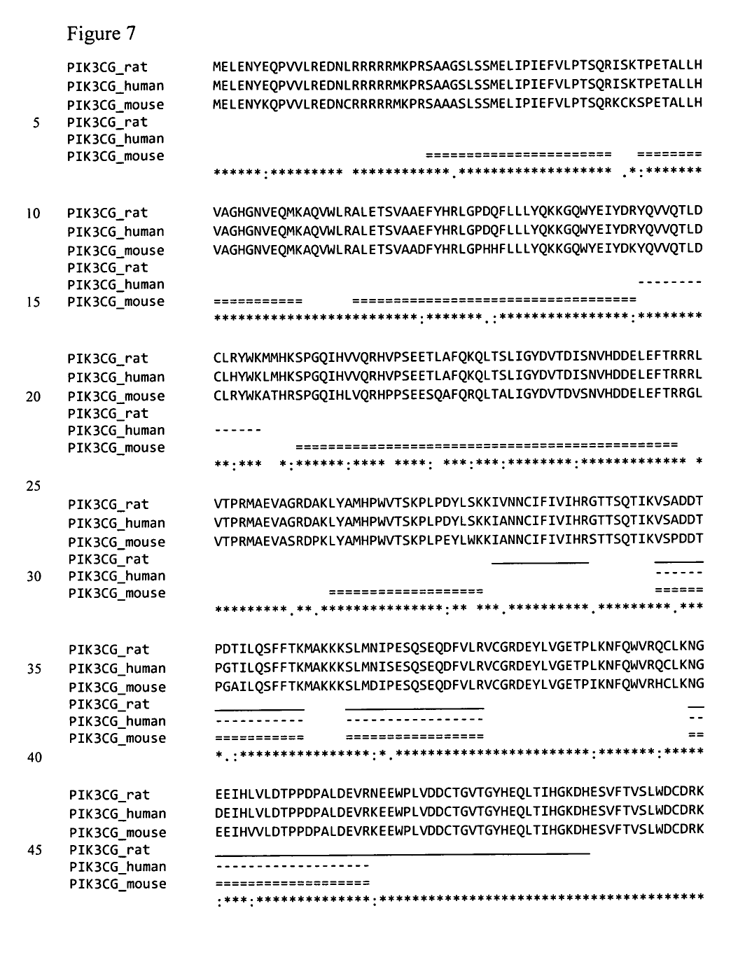

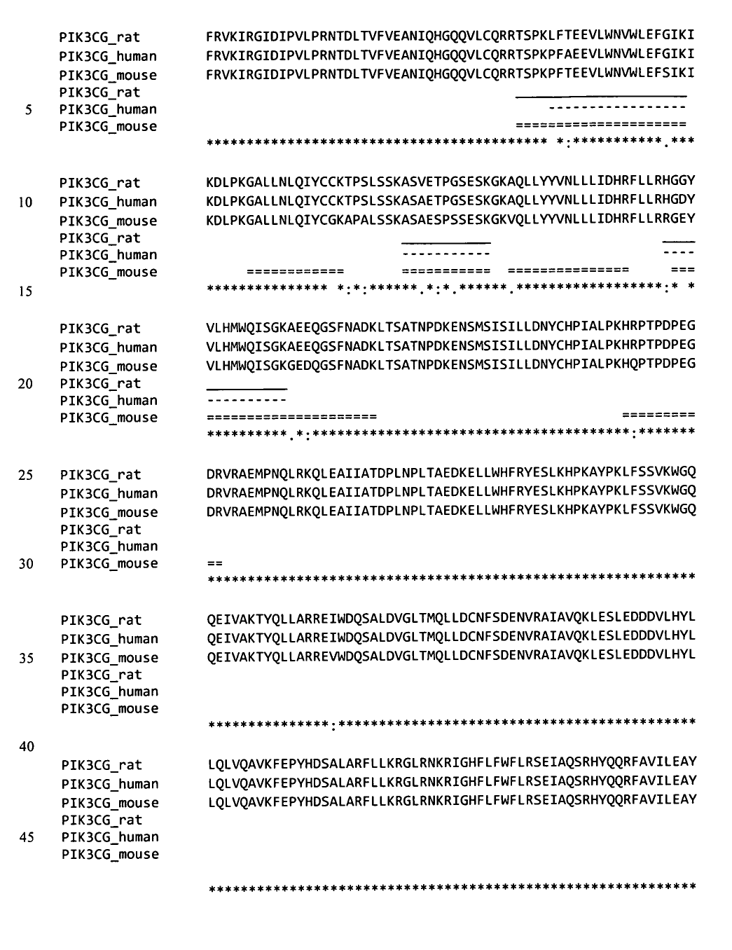

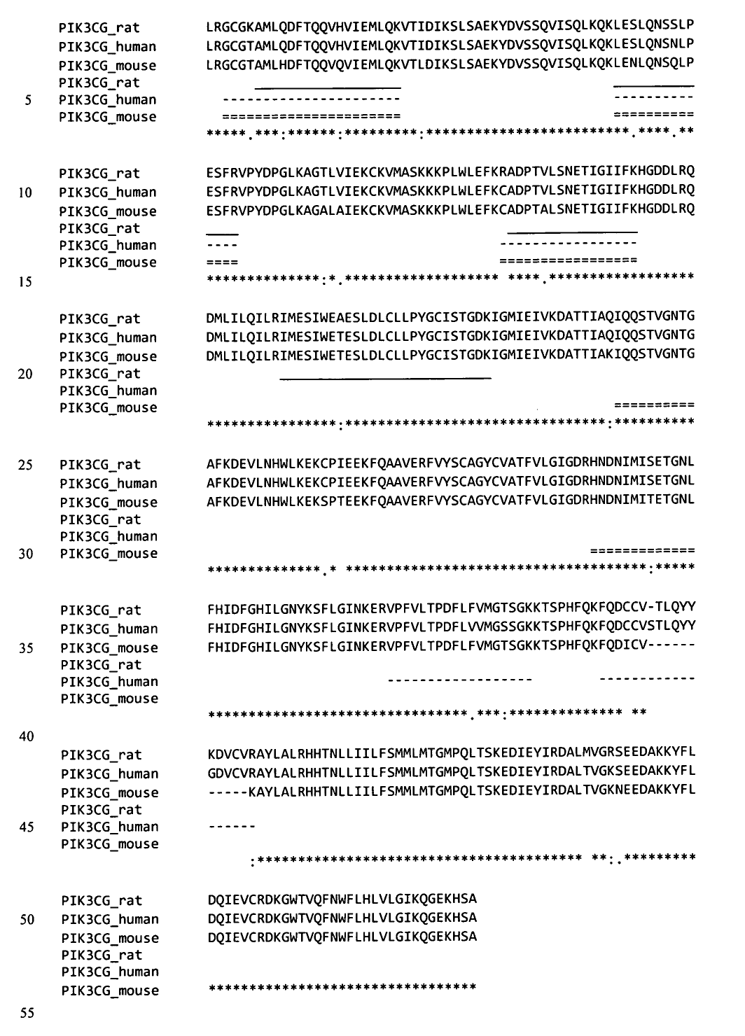

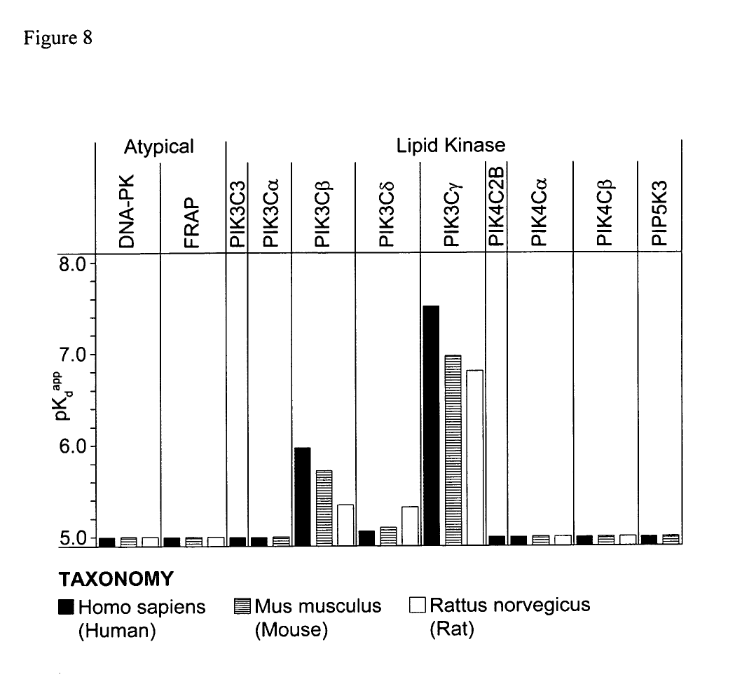

Since compound efficacy and toxicity are evaluated in laboratory animals, it is important to determine its potency and selectivity in the relevant species. In order to minimize inter-assay variation, we used a mass spectrometric targeted data acquisition approach (Savitski et al., 2010. J. Am. Soc. Mass. Spectrom. 21(10):1668-1679) based on species specific proteotypic peptides (FIG. 7) and mixed cell lysates. Competition binding profiles of compound 2 with the affinity matrix were acquired in mixed extracts of human, mouse and rat cell lines. Interestingly, despite the high sequence conservation of the class I PI3K isoforms between human and rodents, the potency of compound 2 for PI3Kγ and PI3Kβ was consistently two- to four-fold lower in mouse and rat compared to human (FIG. 8).

Reagents. All reagents were purchased from Sigma-Aldrich (St. Louis, Mo., USA) unless otherwise noted below. Antibodies were purchased from the following suppliers: anti-mTOR (Cell Signaling Technology, Danvers, Mass., USA; catalogue number 2972), anti-PI3Kδ (Santa Cruz Biotechnology, Santa Cruz, Calif., USA; catalogue number sc-7176), anti-PI3Kγ (Jena Bioscience, Jena, Germany, catalogue number ABD-026L) and anti-DNA-PK (Calbiochem/Merck Chemicals, Nottigham, UK, catalogue number NA57). Secondary antibodies labeled with IRDye®800 were from LI-COR Biosciences (Lincoln, Nebr., USA). The reference compound PI-103 was purchased from Calbiochem (catalogue number 528100).

Cell culture. Jurkat E6.1, HL60, Ramos and HeLa cells were purchased from the American Type Culture Collection (ATCC, Manassas, VA, USA). Jurkat E6.1 cells were cultured in RPMI1640 medium supplemented with 4.5 g/L glucose, 10 mM Hepes, 1 mM sodium pyruvate and 10% Fetal Calf Serum (FCS). Cells were expanded to a maximum density of 10⁶ cells/ml.

Preparation of cell extracts. Frozen cell pellets were homogenized in lysis buffer (50 mM Tris-HCl, 0.8% Igepal®-CA630, 5% glycerol, 150 mM NaCl, 1.5 mM MgCl₂, 25 mM NaF, 1 mM sodium vanadate, 1 mM DTT, pH 7.5). One complete EDTA-free protease inhibitor tablet (Roche Diagnostics, 1 873 580) per 25 ml was added. The sample was dispersed using a Dounce homogenizer, kept rotating for 30 minutes at 4° C. and spun for 10 minutes at 20,000×g at 4° C. The supernatant was spun again for 1 hour at 145,000×g. The protein concentration was determined by Bradford assay (Bio-Rad Laboratories GmbH, Munich, Germany), and aliquots were snap frozen in liquid nitrogen and stored at −80° C.

Proteomics-based inhibitor profiling. Affinity profiling assays were performed as described previously (WO 2009/09802). Derivatized sepharose® beads (35 μl beads per sample) were equilibrated in lysis buffer and incubated with 1 ml (5 mg protein) cell lysate, which had been preincubated with test compound or vehicle for 45 min, on an end-over-end shaker for 1 hour. Incubation was done at 4° C. for all compounds. Beads were transferred to disposable columns (MoBiTec), washed with lysis buffer containing 0.2% NP-40 and eluted with 50 μl 2× SDS sample buffer. Proteins were alkylated with 200 mg/ml iodoacetamide for 30 minutes, separated on 4-12% NuPAGE (Invitrogen), and stained with colloidal Coomassie.

Sample preparation for mass spectrometry. Gels were cut into slices across the entire separation range and subjected to in-gel digestion. Peptide extracts were labeled with tandem mass tags (TMT®) (Thermo-Fisher Scientific) in 40 mM triethylammoniumbicarbonate (TEAB), pH 8.53. After quenching of the reaction with glycin labeled extracts were combined. For compound profiling experiments extracts from vehicle treated samples were labeled with TMT® reagent 131, and combined with extracts from compound-treated samples labeled with TMT® reagents 126-130, optionally fractionated using reversed phase chromatography at pH 12, dried and acidified prior to LC-MS/MS analysis.

LC-MS/MS analysis. Samples were dried in vacuo and resuspended in 0.1% formic acid in water and aliquots of the sample were injected into a nano-LC system (Eksigent 1 D+) coupled to LTQ-Orbitrap mass spectrometers (Thermo-Finnigan). Peptides were separated on custom 50 cm×100 μM (internal diameter) reversed-phase columns (Reprosil) at 40° C. Gradient elution was performed from 2% acetonitrile to 40% acetonitrile in 0.1% formic acid over 2-4 hours. LTQ-Orbitrap XL and Orbitrap Velos instruments were operated with XCalibur 2.0/2.1 software. Intact peptides were detected in the Orbitrap at 30.000 resolution. Internal calibration was performed using the ion signal from (Si(CH₃)₂O)6 H+ at m/z 445.120025 (Olsen, et al., 2005. Mol. Cell. Proteomics 4, 2010-2021). Data-dependent tandem mass spectra were generated for up to six peptide precursors using a combined CID/HCD approach (Kocher, et al., 2009, J. Proteome Res. 8, 4743-4752) using HCD at a resolution of 7,500 with a 70% normalized collision energy. For CID, up to 5,000 ions (Orbitrap XL) or up to 3,000 ions (Orbitrap Velos) were accumulated in the ion trap within a maximum ion accumulation time of 200 msec. For HCD, target ion settings were 50,000 (Orbitrap XL) and 25,000 (Orbitrap Velos), respectively.

Creation of peptide inclusion lists. History mass spectrometry data was queried to find tryptic peptides for the proteins of interest that are only present in mouse or in human proteomes, (or in human or rat proteins depending on the experiment). Subsequently for each human and mouse (or rat) protein that was of interest to the study the selected peptides were ranked according to the quality of the quantification and ease of identification inherent to these peptides (Savitski et al., 2010, J. Am. Soc. Mass. Spectrom. 21(10):1668-1679). The top 40 peptides for each protein (or as many as were available in case 40 was not reached) were selected and their mass over charge values together with retention time windows were fed into the instrument software.

Peptide and protein identification. Raw data were processed with in-house developed software written in Python that utilizes some core elements from the open source Multiplier Python-based environment (Parikh et al., 2009. BMC Bioinformatics 10, 364). Mgf files were created and submitted to the Mascot 2.0 (Matrix Science) search engine (Perkins et al., 1999, Electrophoresis 20, 3551-3567), using 10 p.p.m. mass tolerance for peptide precursors and 0.8 Da (CID) tolerance for fragment ions. Carbamidomethylation of cysteine residues and iTRAQ®/TMT® modification of lysine residues were set as fixed modifications and S,T,Y phosphorylation, methionine oxidation, N-terminal acetylation of proteins and iTRAQ®/TMT® modification of peptide N termini were set as variable modifications. The search database consisted of a customized version of the International Protein Index database combined with a decoy version of this database (Elias and Gygi, 2007, Nat. Methods 4, 207-14) created using a script supplied by Matrix Science. Unless stated otherwise, we accepted protein identifications as follows: (i) for single spectrum to sequence assignments, we required this assignment to be the best match and a minimum Mascot score of 31 and a 10x difference of this assignment over the next best assignment. Based on these criteria, the decoy search results indicated <1% false-discovery rate (FDR); (ii) for multiple spectrum to sequence assignments and using the same parameters, the decoy search results indicate <0.1% FDR. For protein quantification a minimum of two sequence assignments matching to unique peptides was required. FDR for quantified proteins was <<0.1%.

Peptide and protein quantification. Centroided iTRAQ®/TMT® reporter ion signals were computed by the XCalibur software operating and extracted from MS data files using customized scripts. Only peptides unique for identified proteins and present on the inclusion lists were used for relative protein quantification. Reporter ion intensities were multiplied with the ion accumulation time yielding an area value proportional to the number of reporter ions present in the mass analyzer. For compound competition binding experiments, fold-changes are reported based on reporter ion areas in comparison to vehicle control and were calculated using sum-based bootstrap algorithm. Fold-changes were corrected for isotope purity and adjusted for interference caused by co-eluting nearly isobaric peaks as estimated by the signal-to-interference measure peptides (Savitski et al., 2010. J. Am. Soc. Mass. Spectrom. 21(10): 1668-1679). Dose-response curves were fitted using R (http://www.r-project.org/) and the drc package (http://www.bioassay.dk), as described previously (Bantscheff et al., 2007. Nat. Biotechnol. 25, 1035-44). IC₅₀ values were transformed into apparent dissociation constants following described procedures (Sharma et al., 2009. Nature Methods 6, 741-744).

1. A method for the determination of the species profile of a protein-interacting compound, comprising the steps of
a) providing a protein preparation derived from one species (species-1) containing a species-1-variant of said protein,
b) providing a protein preparation derived from another species (species-2) containing a species-2-variant of said protein,
c) mixing said protein preparations,
d) contacting said mixed protein preparations with a ligand of said protein and with a given protein-interacting compound under conditions allowing the formation of a complex between said ligand and said protein,
e) detecting the complexes formed in step d) with the help of mass spectrometry.

2. The method of claim 1, wherein a difference between the amount of complexes formed between the species-1-protein variant and the ligand and the species-2-protein variant and the ligand indicates that the compound has a higher affinity for the species-variant which forms less complexes.

3. The method of claim 1, wherein the amount of the complexes is determined by separating the protein variants from the ligand and subsequent detection of the separated protein variants or subsequent determination of the amount of the separated protein variants.

4. The method of claim 1, wherein detecting of the complexes and/or determining the amount of the complexes is carried out by quantitative mass spectrometry.

5. The method of claim 1, wherein the given compound is selected from the group consisting of synthetic compounds or organic synthetic drugs, more preferably small molecules, organic drugs, or natural small molecule compounds.

6. The method of claim 1, wherein the given compound is an antibody.

7. The method of claim 1, wherein the given compound is an inhibitor of the protein.

8. The method of claim 1, wherein the provision of a protein preparation includes the steps of harvesting at least one cell containing said protein and lysing the cell.

9. The method of claim 1, wherein the steps of the formation of the complex are performed under essentially physiological conditions.

10. The method of claim 1, wherein said species-1 is human.

11. The method of claim 1, wherein said species-2 is a rodent, preferably rat or mouse.

12. The method of claim 1, wherein said protein is an enzyme.

13. The method of claim 1, wherein the ligand is an antibody.

14. The method of claim 1, wherein detecting of the complexes and/or determining the amount of the complexes includes the quantitation of species-specific peptides.


SEQUENCE LISTING (21 sequences)

SEQ ID NO: 1 - protein, 8 aa, Homo sapiens
STTSQTIK

SEQ ID NO: 2 - protein, 11 aa, Homo sapiens
GEDQGSFNADK

SEQ ID NO: 3 - protein, 8 aa, Homo sapiens
AGALAIEK

SEQ ID NO: 4 - protein, 12 aa, Homo sapiens
GALLNLQIYCGK

SEQ ID NO: 5 - protein, 16 aa, Homo sapiens
QKLENLQNSQLPESFR

SEQ ID NO: 6 - protein, 22 aa, Homo sapiens
IQQSTVGNTGAFKDEVLNHWLK

SEQ ID NO: 7 - protein, 15 aa, Homo sapiens
VCGRDEYLVGETPIK

SEQ ID NO: 8 - protein, 8 aa, Homo sapiens
PLPEYLWK

SEQ ID NO: 9 - protein, 17 aa, Homo sapiens
VSPDDTPGAILQSFFTK

SEQ ID NO: 10 - protein, 11 aa, Mus musculus
ASAETPGSESK

SEQ ID NO: 11 - protein, 6 aa, Mus musculus
SEEDAK

SEQ ID NO: 12 - protein, 8 aa, Mus musculus
AGTLVIEK

SEQ ID NO: 13 - protein, 11 aa, Mus musculus
AEEQGSFNADK

SEQ ID NO: 14 - protein, 16 aa, Mus musculus
QKLESLQNSNLPESFR

SEQ ID NO: 15 - protein, 12 aa, Mus musculus
LGPDQFLLLYQK

SEQ ID NO: 16 - protein, 12 aa, Mus musculus
GALLNLQIYCCK

SEQ ID NO: 17 - protein, 12 aa, Mus musculus
HVPSEETLAFQK

SEQ ID NO: 18 - protein, 14 aa, Mus musculus
YQVVQTLDCLHYWK

SEQ ID NO: 19 - protein, 6 aa, Mus musculus
VTIDIK

SEQ ID NO: 20 - protein, 300 aa, Homo sapiens
MELENYKQPVVLREDNCRRRRRMKPRSAAASLSSMELIPIEFVLPTSQRKCKSPETALLHVAGHGNVEQMKAQVWLRALETSVAADFYHRLGPHHFLLLYQKKGQWYEIYDKYQVVQTLDCLRYWKATHRSPGQIHLVQRHPPSEESQAFQRQLTALIGYDVTDVSNVHDDELEFTRRGLVTPRMAEVASRDPKLYAMHPWVTSKPLPEYLWKKIANNCIFIVIHRSTTSQTIKVSPDDTPGAILQSFFTKMAKKKSLMDIPESQSEQDFVLRVCGRDEYLVGETPIKNFQWVRHCLKNG

SEQ ID NO: 21 - protein, 300 aa, Mus musculus
MELENYEQPVVLREDNLRRRRRMKPRSAAGSLSSMELIPIEFVLPTSQRISKTPETALLHVAGHGNVEQMKAQVWLRALETSVAAEFYHRLGPDQFLLLYQKKGQWYEIYDRYQVVQTLDCLHYWKLMHKSPGQIHVVQRHVPSEETLAFQKQLTSLIGYDVTDISNVHDDELEFTRRRLVTPRMAEVAGRDAKLYAMHPWVTSKPLPDYLSKKIANNCIFIVIHRGTTSQTIKVSADDTPGTILQSFFTKMAKKKSLMNIPESQSEQDFVLRVCGRDEYLVGETPLKNFQWVRQCLKNG